(19) United States
(12) Patent Application Publication
(10) Pub. No.: US 2026/0137737 A1
(43) Pub. Date: May 21, 2026

(54) SECRETIN INDUCING BACTERIA

(71) Applicants: BAYLOR COLLEGE OF MEDICINE, Houston, TX (US); BIOGAIA AB, Stockholm (SE)

(72) Inventors: Robert Allen BRITTON, II, Manvel, TX (US); Heather Ann DANHOF, Houston, TX (US); Sara Chloe DI RIENZI, Houston, TX (US); Charlotta LINDVALL BLOM, Landvetter (SE); Pernilla GARBERG, Lidingö (SE); Jonas FAIJERSON SÄLJÖ, Göteborg (SE)

(21) Appl. No.: 19/119,321

(22) PCT Filed: Oct. 12, 2023
(86) PCT No.: PCT/SE2023/051018

§ 371 Date: Apr. 9, 2025

Related U.S. Application Data
(60) Provisional application No. 63/379,317, filed on Oct. 13, 2022.

(30) Foreign Application Priority Data
Oct. 24, 2022 (SE) 2251234-7

(51) Int. Cl. A61K 35/747; A61K 35/00; A61P 1/00; C12Q 1/02

(52) U.S. Cl. CPC A61K 35/747; A61P 1/00; C12Q 1/025; A61K 2035/115

(57) ABSTRACT

The invention relates to the use of a probiotic composition comprising a lactic acid producing bacterial strain, a conditioned medium or microvesicles from the lactic acid producing bacterial strain in treatment or prevention of hyposecretinemia. The lactic acid producing bacterial strain, the conditioned medium and/or the microvesicles is or are capable of inducing release, and optionally production, of secretin locally in a gastrointestinal tract of a subject suffering from hyposecretinemia or having a risk of suffering from hyposecretinemia. The invention also relates to screening of lactic acid producing bacterial strains suitable in treatment or prevention of hyposecretinemia. Secretin

### TECHNICAL FIELD

The present invention generally relates to lactic acid producing bacteria, and in particular to screening such lactic acid producing bacteria for their capability of secretin induction in the gastrointestinal tract of a subject and the use of such lactic acid producing bacteria in the treatment or prevention of hyposecretinemia.

### BACKGROUND

It is well established that interactions between host and gut microbes are fundamental to health and well-being of the host. Intestinal microbiota generate metabolites that provide the host with nutrients but may also be involved in the immune response and in regulation and development of the host's immune system. There is also known today that the intestinal microbiota can influence the gut-brain axis.

The definition of a probiotic is “Live microorganisms that, when administered in adequate amounts, confer a health benefit on the host.” (WHO/FAO, 2002). Lactobacilli and other lactic acid producing bacteria, such as bifidobacteria, are commonly used as probiotics in various types of foods, for example yoghurt. Growth and colonization of harmful microorganisms can be prevented by such lactic acid producing bacteria through their colonization inside the intestinal ecosystem. Consumption of probiotics may also have effect on several different functions in the body, such as positively effecting digestion and supporting and educating the immune system, just to name a few.

Secretin is a gastrointestinal peptide hormone with several local functions, including regulation of gastric acid levels, regulation of pancreatic bicarbonate, regulation of gastric emptying, and regulation and potentiation of pancreatic digestive enzymes as well as regulation of production of the stomach enzyme pepsin.

Secretin deficiency in a subject may lead to several problems due to the important and diverse roles secretin plays in the subject body. To correct such deficiencies, secretin can be administered via intravenous or sublingual routes to subjects in need thereof to compensate for the loss or subnormal secretin levels. However, several side effects are known from such administration, including abdominal discomfort and pain, abdominal cramps, bloating, flushing, nausea, slow heart rate, low blood pressure, sweating, diarrhea, fatigue, fever, headache, hunger, numbness or tingling in extremities, and rash.

There is, thus, a need for a treatment of secretin deficiency that is not marred by the side effects associated with intravenous or sublingual administration of secretin.

### SUMMARY

It is a general objective to screen for lactic acid producing bacterial strains capable of inducing secretin release in the gastrointestinal tract of a subject.

It is another general objective to use such lactic acid producing bacterial strains in treating or preventing hyposecretinemia.

These and other objectives are met by embodiments described herein.

The present invention is defined in the independent claims. Further embodiments of the invention are defined in the dependent claims.

An aspect of the invention relates to a probiotic composition comprising a lactic acid producing bacterial strain, a conditioned medium from a lactic acid producing bacterial strain and/or microvesicles from a lactic acid producing bacterial strain for use in treatment or prevention of hyposecretinemia. The lactic acid producing bacterial strain, the conditioned medium and/or the microvesicles is or are capable of inducing release of secretin locally in a gastrointestinal tract of a subject suffering from hyposecretinemia or having a risk of suffering from hyposecretinemia.

Another aspect of the invention relates to a method for selecting a lactic acid producing bacterial strain. The method comprises contacting ex vivo intestinal enteroid cells, enteroendocrine S cells or gastrointestinal tissue with a lactic acid producing bacterial strain and/or a conditioned medium from the lactic acid producing bacterial strain. The method also comprises detecting any secretin released from the intestinal enteroid cells, the enteroendocrine S cells or the gastrointestinal tissue in response to contact with the lactic acid producing bacterial strain and/or the conditioned medium. The method further comprises selecting the lactic acid producing bacterial strain if the lactic acid producing bacterial strain and/or the conditioned medium is determined to be capable of inducing secretin release from the intestinal enteroid cells, the enteroendocrine S cells or the gastrointestinal tissue.

A further aspect of the invention relates to a method for selecting a lactic acid producing bacterial strain. The method comprises feeding a germ-free subject with a lactic acid producing bacterial strain and/or a conditioned medium from the lactic acid producing bacterial strain. The method also comprises detecting, in a body sample taken from the germ-free subject, any secretin released from gastrointestinal tissue of the subject induced by the lactic acid producing bacterial strain and/or the conditioned medium. The method further comprises selecting the lactic acid producing bacterial strain if secretin released from the gastrointestinal tissue of the germ-free subject induced by the lactic acid producing bacterial strain and/or the conditioned medium is detected.

The present invention is based on the discovery that some lactic acid producing bacterial strains are capable of inducing secretin release in ex vivo models of the human upper gastrointestinal (GI) tract. Such secretin-inducing lactic acid producing bacterial strains are therefore suitable as an alternative to intravenous or sublingual administration of secretin in subjects suffering from hyposecretinemia. The lactic acid producing bacterial strains are thereby capable of inducing secretin release from the subjects'gastrointestinal tract. Such an approach to induce endogenous secretin release by lactic acid producing bacterial strains have the added benefit of not being marred by all the adverse effects of intravenous or sublingual administration of secretin.

### BRIEF DESCRIPTION OF THE DRAWINGS

The embodiments, together with further objects and advantages thereof, may best be understood by making reference to the following description taken together with the accompanying drawings:

FIG. 1. L. reuteri ATCC PTA 6475 induces secretin production and/or release from the human enteroendocrine cell line J2-tetNGN3 cultured as enteroids. Secretin was detected by ELISA in the enteroid cell culture medium after 3 hours of exposing the enteroids to conditioned medium from bacterial cultures of L. reuteri ATCC PTA 6475. No secretin was, however, detected when the enteroids were subjected to LDM4 control medium, i.e., medium that had not previously been in contact with bacterial cells, for the same amount of time.

FIG. 2. L. reuteri ATCC PTA 6475 induces oxytocin production and/or release from the human enteroendocrine cell line J2-tetNGN3 cultured as enteroids. Oxytocin was detected by ELISA in the enteroid cell culture medium after 3 hours of exposing the enteroids to conditioned medium from bacterial cultures of L. reuteri ATCC PTA 6475. No oxytocin was however detected when the enteroids were subjected to LDM4 control medium, i.e., medium that had not previously been in contact with bacterial cells, for the same amount of time.

FIG. 3. L. reuteri ATCC PTA 6475 and L. reuteri ATCC PTA 4659 induce secretin production and/or release from tissue sections obtained from different intestinal regions of the human gastrointestinal tract. Secretin was detected by ELISA in the culture medium after 3 hours of exposing the intestinal tissue sections to conditioned medium from bacterial cultures of L. reuteri ATCC PTA 6475 or L. reuteri ATCC PTA 4659. No secretin, or very low levels of secretin, were however detected when the intestinal tissue sections were subjected to LDM4 control medium, i.e., medium that had not previously been in contact with bacterial cells, for the same amount of time.

### DETAILED DESCRIPTION

The present invention generally relates to lactic acid producing bacteria, and in particular to screening such lactic acid producing bacteria for their capability of secretin induction in the gastrointestinal tract of a subject and the use of such lactic acid producing bacteria in the treatment or prevention of hyposecretinemia.

The present invention is based on the surprising discovery that certain lactic acid producing bacterial strains can induce endogenous secretin release from the gastrointestinal tract of a subject. In more detail, selected lactic acid producing bacterial strains were capable of inducing secretin release in ex vivo models of the human upper gastrointestinal tract. Accordingly, such secretin-inducing lactic acid producing bacterial strains can be used as an alternative to current treatments of hyposecretinemia, i.e., intravenous or sublingual administration of recombinant secretin. Such intravenous or sublingual administration of recombinant secretin is, as mentioned in the background section, associated with several adverse effects. The lactic acid producing bacterial strains selected for their capability of inducing release of endogenous secretin from the gastrointestinal tract are, however, not marred by such adverse effects. This means that lactic acid producing bacterial strains capable of inducing release of endogenous secretin when administered to a subject can be used as an alternative to intravenous or sublingual administration of recombinant secretin to treat or prevent secretin deficiency but without the shortcomings in terms of adverse or side-effects caused by the intravenous or sublingual administration of recombinant secretin.

The present invention therefore relates to screening for lactic acid producing bacterial strains that are capable of inducing release of secretin from the gastrointestinal tract of a subject and that are therefore suitable for use in treatment or prevention of hyposecretinemia.

An aspect of the embodiments relates to a method for selecting a lactic acid producing bacterial strain. The method comprises contacting ex vivo intestinal enteroid cells, enteroendocrine S cells or gastrointestinal tissue with a lactic acid producing bacterial strain and/or a conditioned medium from the lactic acid producing bacterial strain. The method also comprises detecting any secretin released from the intestinal enteroid cells, the enteroendocrine S cells or the gastrointestinal tissue in response to contact with the lactic acid producing bacterial strain and/or the conditioned medium. The method further comprises selecting the lactic acid producing bacterial strain if the lactic acid producing bacterial strain and/or the conditioned medium is determined to be capable of inducing secretin release from the intestinal enteroid cells, the enteroendocrine S cells or the gastrointestinal tissue.

The lactic acid producing bacterial strain is, thus, selected if the lactic acid producing bacterial strain and/or the conditioned medium is determined to be capable of inducing secretin release from the intestinal enteroid cells, the enteroendocrine S cells or the gastrointestinal tissue. Hence, secretin induced by the lactic acid producing bacterial strain and/or the conditioned medium can thereby be detected in a medium, typically a culture medium, comprising the intestinal enteroid cells, the enteroendocrine S cells or the gastrointestinal tissue.

The method is an ex vivo method of screening lactic acid producing bacterial strains for their capability of inducing release of secretin. The ex vivo screening method contacts intestinal enteroid cells, enteroendocrine S cells or gastrointestinal tissue with the lactic acid producing bacterial strain and/or the conditioned medium from the lactic acid producing bacterial strain. Intestinal enteroid, or intestinal organoid, as used herein refers to a multilobulated structure with a lumen that develops from intestinal stem cells (cycling crypt base columnar cells and quiescent stem cells) near the bottom of the intestinal crypts (also termed the intestinal stem cell niche), or single intestinal stem cells by formation of budding crypts. Such intestinal enteroids have been generated from, among others, mouse and human intestinal stem cells. For instance, the growth of these intestinal stem cells is regulated mainly by Wnt, Notch, epidermal growth factors (EGFs), and the bone morphogenetic proteins (BMPs) signaling pathways in vivo. Wnt and Notch signaling pathways play major roles in the proliferation of stem cells. EGF signals exert the robust mitogenic effects on stem cells via their corresponding receptors (EGFRs). BMP has an inhibitory effect on the stemness. Noggin promotes crypt like structures to form along the flanks of the villi. To generate enteroids, the intestinal crypts containing the intestinal stem cell niches are separated from intestinal tissues by ethylenediaminetetraacetic acid (EDTA) treatment. These crypts are then embedded in, for instance, Matrigel, followed by supplementing with stemness supporting factor cocktails such as EGF, R-spondin-1, Noggin, and Wnt3a. The crypts will gradually develop into 3D enteroids displaying many important organizations of the normal intestinal epithelium. Secretin releasing cells, i.e., so called enteroendocrine S cells, are found throughout the length of the small intestinal mucosa with a decreasing frequency from the duodenum towards the ileum. The intestinal enteroid cells used in this ex vivo could be from any part of the gastrointestinal tract comprising enteroendocrine S cells, such as duodenal enteroid cells or jejunal enteroid cells. Jejunal enteroid cells are then enteroid cells from the middle section of the small intestine (jejunum) and the normal jejunal epithelium, whereas duodenal enteroid cells are enteroid cells from the first section of the small intestine (duodenum) and the normal duodenal epithelium.

In an embodiment, the intestinal enteroid cells are mammalian intestinal enteroid cells, such as mouse, rat, pig or human intestinal enteroid cells, preferably human intestinal enteroid cells.

As an alternative of using intestinal enteroid cells comprising enteroendocrine S cells among other types of cells, the ex vivo method could instead use pure enteroendocrine S cells, such as a population of enteroendocrine S cells or an enteroendocrine S cell line. In such an embodiment, the enteroendocrine S cells are preferably mammalian S cells, such as mouse, rat, pig or human S cells, more preferably human S cells.

The gastrointestinal tissue that can be used in the screening method could be whole gastrointestinal tissue comprising stomach, the small intestine (duodenum, jejunum and ileum) and the large intestine (colon). In an embodiment, the gastrointestinal tissue comprises the whole or at least a portion of the small intestine, such as the duodenum, jejunum and ileum, the duodenum and jejunum, or the jejunum and ileum. It is also possible to use the whole or at least a portion of the duodenum, the jejunum or the ileum, preferably the whole or at least a portion of the duodenum or the jejunum. In these embodiments, the gastrointestinal tissue is preferably a mammalian gastrointestinal tissue, such as mouse, rat, pig or human gastrointestinal tissue, preferably human gastrointestinal tissue.

A conditioned medium, sometimes also referred to as a conditioned culture medium, is a (cell-free culture) medium, in which lactic acid producing bacterial cells have been cultured in for a period of time. As an example, the lactic acid producing bacteria “condition” the medium by releasing or secreting various components or molecules, such as proteins, peptides, enzymes, cytokines, chemokines, and/or chemicals into the medium. The conditioned medium could then be obtained, for instance, by centrifugation to obtain the conditioned medium as the resulting cell-free supernatant. Conditioned medium as used herein also encompass, such culture medium following one or more processing steps, such as filtration, concentration, sterilization, etc.

The conditioned medium then comprises metabolites, such as proteins or peptides, and other structures released by the lactic acid producing bacteria during culture, such as microvesicles.

An example of processing a conditioned medium could be to concentrate or retrieve microvesicles, such as by centrifugation, to obtain a conditioned medium with concentrated microvesicles. Hence, in such an embodiment, the conditioned medium mainly comprises the microvesicle fraction(s).

Microvesicles (MVs, μV), also referred to as, for instance, membrane vesicles, outer membrane vesicles, extracellular vesicles in the art, are a demonstrated form of communication used by bacteria and eukaryotic cells.

The contacting step of the screening method could be in terms of contacting the intestinal enteroid cells with the lactic acid producing bacterial strain and/or the conditioned medium, such as by adding the lactic acid producing bacterial strain and/or the conditioned medium to the intestinal enteroid cells in a culture dish or in a culture matrix, such as Matrigel, comprising the intestinal enteroid cells.

In another embodiment, the contacting step of the screening method could be in terms of contacting the S cells with the lactic acid producing bacterial strain and/or the conditioned medium, such as by adding the lactic acid producing bacterial strain and/or the conditioned medium to the enteroendocrine S cells in a culture dish.

In the case of a gastrointestinal tissue, the gastrointestinal tissue may be attached to a first tubing in a first end of the gastrointestinal tissue and to a second tubing in a second end of the gastrointestinal tissue. In such a case, the lactic acid producing bacterial strain and/or the conditioned medium could contact the gastrointestinal tissue by adding the lactic acid producing bacterial strain and/or the conditioned medium to the first tubing. In another embodiment, at least one gastrointestinal tissue section is contacted with the lactic acid producing bacterial strain and/or the conditioned medium by submerging the gastrointestinal tissue section(s) into a culture medium comprising the lactic acid producing bacterial strain and optionally the conditioned medium, or submerging the gastrointestinal tissue section(s) into the conditioned medium. In an embodiment, the contacting step comprises contacting ex vivo the intestinal enteroid cells, the enteroendocrine S cells or the gastrointestinal tissue with the lactic acid producing bacterial strain. In another embodiment, the contacting step comprises contacting ex vivo the intestinal enteroid cells, the enteroendocrine S cells or the gastrointestinal tissue with the conditioned medium from the lactic acid producing bacterial strain. In a further embodiment, the contacting step comprises contacting ex vivo the intestinal enteroid cells, the enteroendocrine S cells or the gastrointestinal tissue with both the lactic acid producing bacterial strain and the conditioned medium from the lactic acid producing bacterial strain.

The detection of whether the lactic acid producing bacterial strain and/or the conditioned medium is capable of inducing secretin production and release from the intestinal enteroid cells, the enteroendocrine S cells or the gastrointestinal tissue can be performed according to various embodiments. For instance, a sample could be taken from the culture medium comprising the intestinal enteroid cells, the enteroendocrine S cells or the gastrointestinal tissue and the added lactic acid producing bacterial strain and/or the conditioned medium and then analyzed for the presence of secretin. Correspondingly, a sample could be taken from the above-mentioned second tubing connected to the gastrointestinal tissue. In either case, the sample is analyzed for the presence of secretin. The analysis of the presence of any secretin in the sample can, as an example, be performed using a secretin enzyme-linked immunosorbent assay (ELISA) or by mass spectrometry (MS).

In an embodiment, the method also comprises detecting any secretin released from the intestinal enteroid cells, the enteroendocrine S cells or the gastrointestinal tissue prior to contact with the lactic acid producing bacterial strain and/or the conditioned medium. In this embodiment, the method also comprises comparing an amount of secretin released from the intestinal enteroid cells, the enteroendocrine S cells or the gastrointestinal tissue in response to contact with the lactic acid producing bacterial strain and/or the conditioned medium with an amount of secretin released from the intestinal enteroid cells, the enteroendocrine S cells or the gastrointestinal tissue prior to contact with the lactic acid producing bacterial strain and/or the conditioned medium. In such a case, the method further comprises selecting the lactic acid producing bacterial strain if the amount of secretin released from the intestinal enteroid cells, the enteroendocrine S cells or the gastrointestinal tissue in response to contact with the lactic acid producing bacterial strain and/or the conditioned medium is higher than the amount of secretin released from the intestinal enteroid cells, the enteroendocrine S cells or the gastrointestinal tissue prior to contact with the lactic acid producing bacterial strain and/or the conditioned medium.

In another embodiment, the method also comprises detecting any secretin released from the intestinal enteroid cells, the enteroendocrine S cells or the gastrointestinal tissue in contact with a reference medium lacking the lactic acid producing bacterial strain and the conditioned medium. In this embodiment, the method also comprises comparing an amount of secretin released from the intestinal enteroid cells, the enteroendocrine S cells or the gastrointestinal tissue in response to contact with the lactic acid producing bacterial strain and/or the conditioned medium with an amount of secretin released from the intestinal enteroid cells, the enteroendocrine S cells or the gastrointestinal tissue in response to contact with the reference medium. In such a case, the method further comprises selecting the lactic acid producing bacterial strain if the amount of secretin released from the intestinal enteroid cells, the enteroendocrine S cells or the gastrointestinal tissue in response to contact with the lactic acid producing bacterial strain and/or the conditioned medium is higher than the amount of secretin released from the intestinal enteroid cells, the enteroendocrine S cells or the gastrointestinal tissue in response to contact with the reference medium.

The reference medium is, in an embodiment, a culture medium, in particular a same type of culture medium used to culture the lactic acid producing bacterial strain and thereby to produce the conditioned medium. However, this reference medium does not comprise any lactic acid producing bacterial strain or any conditioned medium from the lactic acid producing bacterial strain. This means that the lactic acid producing bacterial strain has not been in contact with the reference medium.

In a particular embodiment, the lactic acid producing bacterial strain is selected as being effective in inducing release of secretin if the amount of secretin released from the intestinal enteroid cells, enteroendocrine S cells or gastrointestinal tissue in response to contact with the lactic acid producing bacterial strain and/or the conditioned medium is at least 110%, preferably at least 120%, and more preferably at least 150% of the amount of secretin released from the intestinal enteroid cells, enteroendocrine S cells or gastrointestinal tissue in response to contact with the reference medium. The intestinal enteroid cells, as shown in FIG. 1, or enteroendocrine S cells or gastrointestinal tissue, might not release any detectable amount of secretin at all in response to contact with the reference medium. In such a case, the lactic acid producing bacterial strain is selected as being effective in inducing release of secretin if detectable amounts of secretin are released from the intestinal enteroid cells, enteroendocrine S cells or gastrointestinal tissue in response to contact with the lactic acid producing bacterial strain and/or the conditioned medium. In a further particular embodiment, the lactic acid producing bacterial strain is selected as being effective in inducing release of secretin if the amount or concentration of secretin released from the intestinal enteroid cells, enteroendocrine S cells or gastrointestinal tissue in response to contact with the lactic acid producing bacterial strain and/or the conditioned medium is above a threshold amount or concentration. This threshold amount or concentration could be determined based on the amount or concentration of secretin released from the intestinal enteroid cells, enteroendocrine S cells or gastrointestinal tissue in response to contact with the reference medium.

In an embodiment, the lactic acid producing bacterial strain and/or the conditioned medium is not only capable of inducing release of secretin from the intestinal enteroid cells, the enteroendocrine S cells or the gastrointestinal tissue but also production of secretin. In such an embodiment, the method comprises detecting any secretin produced by and released from the intestinal enteroid cells, the enteroendocrine S cells or the gastrointestinal tissue in response to contact with the lactic acid producing bacterial strain and/or the conditioned medium. The method also comprises selecting the lactic acid producing bacterial strain if the lactic acid producing bacterial strain and/or the conditioned medium is determined to be capable of inducing secretin production by and release from the intestinal enteroid cells, the enteroendocrine S cells or the gastrointestinal tissue.

In an embodiment, the screening method comprises selecting the lactic acid producing bacterial strain as suitable for use in treatment or prevention of hyposecretinemia if the lactic acid producing bacterial strain and/or the conditioned medium is determined to be capable of inducing secretin release from the intestinal enteroid cells, the enteroendocrine S cells or the gastrointestinal tissue.

The above-described screening method and embodiments thereof is an ex vivo method for identification and selection of lactic acid producing bacteria as effective in inducing secretin release. The invention is, however, not limited thereto. The screening method could alternatively be performed as an in vivo method. Another aspect of the invention therefore relates to a method for selecting a lactic acid producing bacterial strain. The method comprises feeding a germ-free subject with a lactic acid producing bacterial strain and/or a conditioned medium from the lactic acid producing bacterial strain. The method also comprises detecting, in a body sample taken from the subject, any secretin released from gastrointestinal tissue of the subject induced by the lactic acid producing bacterial strain and/or the conditioned medium. The method further comprises selecting the lactic acid producing bacterial strain if secretin released from the gastrointestinal tissue of the subject induced by the lactic acid producing bacterial strain and/or the conditioned medium is detected.

Hence, in this method a test subject is a germ-free (GF) subject that lacks or is deficient of gastrointestinal microbiota. The GF subject is a non-human GF animal subject and preferably selected from the group consisting of mouse, rat, guinea pig, pig, cat, dog, sheep, horse, non-human primate, monkey, or bird, and more preferably pig, mouse or rat.

As an example, GF piglets could be used as test subject in this in vivo screening method. In such a case, the lactic acid bacterial strain and/or conditioned medium could be administered enterally to the GF piglets. In another example, GF mice are used.

The bacteria of the lactic acid producing bacterial strain to be tested in the method are fed, i.e., inoculated, such as gavaged, to the GF subject, such as in the form of a solubilized powder, capsule, or tablet comprising the bacteria or a solution comprising the bacteria. Alternatively, or in addition, the cell-free, conditioned medium from the lactic acid producing bacterial strain is fed, i.e., inoculated, such as gavaged, to the GF subject, such as in the form a solution.

The body sample could be any sample taken from the subject and that would normally contain secretin. For instance, the sample could be a gastrointestinal sample, a tissue sample taken from the subject, such as from gastrointestinal tissue, or a body fluid sample, such as a blood sample, a plasma sample, a serum sample or a duodenal aspirate. In a particular embodiment, the body sample is a body fluid sample, preferably a blood sample, a plasma sample, a serum sample or a duodenal aspirate. Analysis of the presence or amount of secretin the body sample can be performed as described in the foregoing, such as using a secretin ELISA or MS.

In an embodiment, the method also comprises detecting, in a body sample taken from the germ-free subject, any secretin released from the gastrointestinal tissue of the subject prior to feeding the germ-free subject with the lactic acid producing bacterial strain and/or the conditioned medium. In such an embodiment, the method further comprises comparing an amount of secretin released from the gastrointestinal tissue of the subject induced by the lactic acid producing bacterial strain and/or the conditioned medium with an amount of secretin released from the gastrointestinal tissue of the germ-free subject prior to feeding the germ-free subject with the lactic acid producing bacterial strain and/or the conditioned medium. In such a case, the method comprises selecting the lactic acid producing bacterial strain if the amount of secretin released from the gastrointestinal tissue of the subject induced by the lactic acid producing bacterial strain and/or the conditioned medium is higher than the amount of secretin released from the gastrointestinal tissue of the germ-free subject prior to feeding the germ-free subject with the lactic acid producing bacterial strain and/or the conditioned medium.

In another embodiment, the method also comprises feeding a germ-free reference subject with a reference medium lacking the lactic acid producing bacterial strain and the conditioned medium from the lactic acid producing bacterial strain. The method further comprises detecting, in a body sample taken from the germ-free reference subject, any secretin released from the gastrointestinal tissue of the reference subject induced by the reference medium. In such an embodiment, the method further comprises comparing an amount of secretin released from the gastrointestinal tissue of the subject induced by the lactic acid producing bacterial strain and/or the conditioned medium with an amount of secretin released from the gastrointestinal tissue of the germ-free reference subject induced by the reference medium. In such a case, the method comprises selecting the lactic acid producing bacterial strain if the amount of secretin released from the gastrointestinal tissue of the subject induced by the lactic acid producing bacterial strain and/or the conditioned medium is higher than the amount of secretin released from the gastrointestinal tissue of the germ-free reference subject induced by the reference medium.

In a particular embodiment, the lactic acid producing bacterial strain is selected as being effective in inducing release of secretin if the amount of secretin released from the gastrointestinal tissue induced by the lactic acid producing bacterial strain and/or the conditioned medium is at least 110%, preferably at least 120%, and more preferably at least 150% of the amount of secretin released from the gastrointestinal tissue of the germ-free reference subject induced by the reference medium. In some cases, the gastrointestinal tissue of the germ-free reference subject might not release any detectable amount of secretin at all as induced by the reference medium. In such a case, the lactic acid producing bacterial strain is selected as being effective in inducing release of secretin if detectable amounts of secretin are released from the gastrointestinal tissue induced by the lactic acid producing bacterial strain and/or the conditioned medium. In a further particular embodiment, the lactic acid producing bacterial strain is selected as being effective in inducing release of secretin if the amount or concentration of secretin released from the gastrointestinal tissue induced by the lactic acid producing bacterial strain and/or the conditioned medium is above threshold amount or concentration. This threshold amount or concentration can be defined based the amount of secretin released from the gastrointestinal tissue of the germ-free reference subject induced by the reference medium.

In an embodiment, the lactic acid producing bacterial strain and/or the conditioned medium is not only capable of inducing release of secretin from the gastrointestinal tissue but also production of secretin. In such an embodiment, the method comprises detecting, in the body sample taken from the subject, any secretin produced by and released from gastrointestinal tissue of the subject induced by the lactic acid producing bacterial strain and/or the conditioned medium. The method further comprises selecting the lactic acid producing bacterial strain if secretin produced by and released from the gastrointestinal tissue of the subject induced by the lactic acid producing bacterial strain and/or the conditioned medium is detected.

In an embodiment, the method comprises selecting the lactic acid producing bacterial strain as suitable for use in treatment or prevention of hyposecretinemia if secretin released from the gastrointestinal tissue of the subject induced by the lactic acid producing bacterial strain and/or the conditioned medium is detected.

In an embodiment, the lactic acid producing bacterial strain tested in the above-described ex vivo (in vitro) or in vivo screening methods is a Lactobacillus bacterial strain. In a particular embodiment, the Lactobacillus bacterial strain a Lactobacillus reuteri bacterial strain.

The above-described screening methods can be used to identify and select lactic acid producing bacterial strains that are capable of inducing release of secretin locally in the gastrointestinal tract of a subject. Such a selected lactic acid producing bacterial strain is then useful in the treatment or prevention of hyposecretinemia, i.e., secretin deficiency.

An aspect of the invention, thus, relates to a probiotic composition comprising a lactic acid producing bacterial strain, a conditioned medium from a lactic acid producing bacterial strain and/or microvesicles from a lactic acid producing bacterial strain. The lactic acid producing bacterial strain, the conditioned medium and/or the microvesicles is or are capable of inducing release of secretin locally in a gastrointestinal tract of a subject. The probiotic composition is for use in treatment or prevention of hyposecretinemia in a subject suffering from hyposecretinemia or, in the case of prophylactic treatment, having a risk of suffering from hyposecretinemia.

Normally, when food reaches the stomach, hydrochloric acid (HCl), also called gastric acid, gastric juice or stomach acid when present in the GI tract, helps to digest the food in order to facilitate extraction of nutrients further down in the GI tract. Hydrochloric acid is highly acidic, and the normal pH of hydrochloric acid in the stomach is between 1 and 3. In those suffering from low stomach acid production in gastric secretions of the stomach, also called hypochlorhydria, the pH is usually between 3 and 5. Achlorhydria on the other hand is the clinical condition where such stomach acid production is absent and it usually presents with a stomach pH of over 5.

Hydrochloric acid is induced in the stomach after a meal, and it is also the signal that normally induces production and release of secretin in the upper GI tract. In a subject suffering from achlorhydria or hypochlorhydria, hydrochloric acid is absent or present at subnormal levels, which thus results in absent or reduced levels of secretin production and release.

When hydrochloric acid passes from the stomach into the duodenum, and further into the small intestine, secretin is released locally in the gastrointestinal system by S-cells of the gastrointestinal mucosal epithelium. The released secretin can then either act locally or enter the bloodstream. Through the bloodstream, the so-released secretin stimulates duct cells of the pancreas to secrete water and bicarbonate. By this secretin-regulated mechanism, the hydrochloric acid is promptly diluted and neutralized, preventing damage to the intestinal lining. This secretin-mediated neutralization of the pH also improves the effect of pancreatic digestive enzymes released into the duodenum to digest sugars, starch, and fat. In addition, secretin has a direct effect on pancreatic acinar cells, which potentiates digestive enzyme secretion from the pancreas into the GI tract, which are involved in the digestion of sugars, fats, and starches. Secretin is also involved in regulating the secretion of the stomach enzyme pepsin, which is responsible for protein digestion. These pancreatic and stomach enzymes are, thus, pivotal for normal digestion and if secretin is not present, or present at subnormal levels, this will significantly disrupt digestion. In addition, secretin has an effect on the rate of gastric emptying, and any disturbances in gastric emptying, such as those that would occur if secretin is absent or present at subnormal levels, results in a dysfunctional digestion.

Since secretin is induced in the upper GI tract by a reduction in pH due to entering of acid from the stomach, reduced levels of hydrochloric acid, such as in subjects suffering from achlorhydria or hypochlorhydria, results in subnormal levels of secretin in the GI tract. This, in turn, attenuates or blocks all the effects normally induced or triggered by secretin in the GI tract, including the rate of gastric emptying, the potentiation of digestive enzyme secretion from the pancreas, and pH optimization for digestive enzyme activity, ultimately leading to dysfunctional digestion.

Achlorhydria or hypochlorhydria can be due to several reasons, including age, use of acid-suppressing medications, such as proton pump inhibitors (PPIs) and histamine receptor (H2) blockers, also referred to as H2 antagonists, chronic gastritis, atrophic gastritis, chronic stress, bacterial infection, alcohol overconsumption, and vitamin deficiency. Also, surgical procedures can negatively affect the gastric acid production in subjects leading to achlorhydria or hypochlorhydria. Hence, subjects that have undergone a gastric surgery and/or vagotomy may also suffer from achlorhydria or hypochlorhydria.

Hyposecretinemia or secretin deficiency as used herein relates to a condition causing subnormal secretin production and/or release, and thereby subnormal secretin levels following food intake. As discussed above, such subnormal secretin production and/or release causes problems with gastric emptying, digestive enzyme secretion from the pancreas, and pH optimization for digestive enzyme activity, which ultimately leads to dysfunctional digestion. Hence, a particular example of a condition associated with secretin deficiency is dysfunctional digestion, also referred to as indigestion.

An embodiment therefore relates to a probiotic composition comprising the lactic acid producing bacterial strain, the conditioned medium and/or the microvesicles capable of inducing release of secretin locally in a gastrointestinal tract of a subject for use in treatment or prevention of dysfunctional digestion in a subject suffering from dysfunctional digestion or having the risk of suffering from dysfunctional digestion.

In an embodiment, the hyposecretinemia is caused by achlorhydria or hypochlorhydria. In such an embodiment, the probiotic composition is for use in treatment or prevention of hyposecretinemia in a subject suffering from achlorhydria or hypochlorhydria or having the risk of suffering from achlorhydria or hypochlorhydria. The lactic acid producing bacterial strain, the conditioned medium and/or the microvesicles is or are then capable of inducing release of secretin locally in the gastrointestinal tract of a subject suffering from hyposecretinemia caused by achlorhydria or hypochlorhydria.

As mentioned in the foregoing, the subject may suffer from dysfunctional digestion, which may be caused by the achlorhydria or hypochlorhydria resulting in subnormal production and/or release of secretin. Hence, in such an embodiment, the subject is suffering from achlorhydria-dependent or hypochlorhydria-dependent dysfunctional digestion.

An embodiment therefore relates to a probiotic composition comprising the lactic acid producing bacterial strain, the conditioned medium and/or the microvesicles capable of release of secretin locally in a gastrointestinal tract of a subject for use in treatment or prevention of achlorhydria-dependent or hypochlorhydria-dependent dysfunctional digestion in a subject suffering from achlorhydria-dependent or hypochlorhydria-dependent dysfunctional digestion or having the risk of suffering from such achlorhydria-dependent or hypochlorhydria-dependent dysfunctional digestion.

Age is one of the most prevalent factors that contribute to a reduction in stomach acid production and/or secretion. In particular, persons over the age of 60 generally experience a significant reduction in stomach acid production and/or secretion.

Hence, in a particular embodiment, the subject is an elderly subject suffering from achlorhydria or hypochlorhydria, such as achlorhydria-dependent or hypochlorhydria-dependent dysfunctional digestion. Elderly subject as used herein refers to a human subject at age equal to or above 60 years, preferably equal to or above 65 years, and more preferably equal to or above 70 years.

Acid-suppressing medications, such as PPIs and H2 blockers, may be taken to treat various conditions including, but not limited to, esophagitis, gastroesophageal reflux disease (GERD), nonerosive reflux disease (NERD), peptic ulcer disease (PUD), nonsteroidal anti-inflammatory drug (NSAID) associated ulcers, Zollinger-Ellison syndrome (ZES), functional dyspepsia and/or Helicobacter pylori infection. However, taking such PPIs or H2 blockers may lead to hypochlorhydria and, in the extreme case, achlorhydria, with the following adverse effects of subnormal or absent gastric acid production and/or secretion.

Illustrative, but non-limiting, examples of PPIs include omeprazole, lansoprazole, pantoprazole, raberprazole, esomeprazole, dexlanosoprazole and illustrative, but non-limiting, examples of H2 blockers include cimetidine, ranitidine, nizatidine, and/or famotidine.

Accordingly, the subject may be taking, such as prescribed, a medication for lowering or suppressing gastric acid production and/or release, preferably a PPI and/or a H2 blocker. The probiotic composition may then be used in treatment or prevention of adverse effects associated with taking PPI and/or H2 blocker. Such adverse effects associated with taking PPI and/or H2 blocker are due to the acid-suppressing effect of the PPIs and H2 blockers, causing hypochlorhydria or achlorhydria, which in turn may lead to hypochlorhydria-dependent or achlorhydria-dependent dysfunctional digestion.

A particular example of subjects that are taking such acid-suppressing medications, in particular PPIs and H2 blockers, are pregnant women, which are taking or prescribed such medications to treat or prevent heartburn during pregnancy.

Also, other conditions than age and PPI and H2 blocker medication may cause hyposecretinemia by reducing gastric acid production and/or release, i.e., may cause hypochlorhydria or achlorhydria. Hence, in an embodiment, the subject is suffering from a condition causing achlorhydria and/or hypochlorhydria. In a particular embodiment, the condition is selected from the group consisting of chronic gastritis, atrophic gastritis, chronic stress, bacterial infection, alcohol overconsumption, or vitamin deficiency. Alternatively, or in addition, the subject has undergone a gastric surgery and/or a vagotomy.

The absent or subnormal secretin levels in subjects suffering from hyposecretinemia, such as due to hypochlorhydria or achlorhydria, may not only manifest itself in dysfunctional digestion but also malnutrition. Such a malnutrition is at least partly caused by absent or subnormal release of digestive enzymes from the pancreas and increased gastric emptying. Accordingly, the gastrointestinal tract will then not efficiently digest the food and will not efficiently take up nutrients therefrom. Hence, in a particular embodiment, the subject is suffering from malnutrition.

The lactic acid producing bacterial strains, the conditioned medium and the microvesicles of the invention are thereby capable of inducing secretin locally in the gastrointestinal track of a subject suffering from absent or reduced levels of secretin. In a typical case, the subject has a functional basic machinery for producing and releasing secretin locally in the gastrointestinal tract. However, the subject lacks the endogenous signaling mechanism for inducing production and/or release of secretin, such as due to an underlying achlorhydria or hypochlorhydria. The lactic acid producing bacterial strains, the conditioned medium and the microvesicles of the invention will then trigger or activate this mechanism to induce release of secretin locally in the gastrointestinal tract even if the subject is suffering from achlorhydria or hypochlorhydria.

The lactic acid producing bacterial strains, the conditioned medium and the microvesicles of the invention are thereby capable of increasing the secretin levels in the gastrointestinal tract of the subject and may even restore the secretin levels from absent or subnormal to normal levels. The increased or normalized secretin levels thereby activate the downstream effects of secretin in the subject, including release of pancreatic enzymes and normalized gastric emptying, leading to normal digestion of food.

The inventors have also surprisingly discovered that secretin induced locally in the gastrointestinal tract by the lactic acid producing bacterial strains, the conditioned medium and/or the microvesicles causes local gastrointestinal release of oxytocin. The inventors found evidence for the direct effect of bacterially-induced secretin on oxytocin production and/or release by blocking the secretin receptor. When secretin binding to this receptor was blocked, no release of oxytocin was observed, thus showing the direct connection between secretin and oxytocin production and/or release.

Oxytocin is a peptide hormone and a neuropeptide, which is normally produced in the hypothalamus and released into the bloodstream by the posterior pituitary. It has many health effects and plays a role in securing emotional bonds between mother and child (social bonding), in reproduction, childbirth, as well as uterus contractions, breastfeeding, and pain relief after childbirth. Oxytocin has also been associated with other functions such as promoting hair and skin growth.

Until now it was not known that lactic acid producing bacterial strains could be involved in oxytocin production or oxytocin release from enterocytes locally in the epithelium of the gastrointestinal tract. The findings by the inventors confirm that oxytocin is produced in and/or released from the gut epithelium in the absence of signals from the lamina propria. With this new knowledge, the inventors propose to use a lactic acid producing bacterial strain, which is capable of inducing release of secretin from enteroendocrine S cells locally in the gastrointestinal tract, to also increase the local release of, or production and release of, oxytocin in the gastrointestinal tract. Oxytocin released locally in the gut may have local effects in the gastrointestinal tract, but it can also be transported away from the gastrointestinal tract via the systemic circulation to exert its effects in other parts of the body.

Accordingly, it is a further aspect and object of the invention to use a probiotic composition comprising a lactic acid producing bacterial strain, a conditioned medium from a lactic acid producing bacterial strain and/or microvesicles produced by a lactic acid producing bacterial strain, which is or are capable of inducing release of secretin, to prevent or treat conditions caused by subnormal levels of oxytocin, such as connection or bonding problems between mother and child, generally referred to as child attachment disorder (CAD), autism, and/or stress-induced breast-feeding problems, also referred to as milk ejection reflex impairment, or breastfeeding with dysphoric milk ejection reflect (D-MER).

A further aspect, thus, relates to a probiotic composition comprising a lactic acid producing bacterial strain, a conditioned medium from a lactic acid producing bacterial strain and/or microvesicles produced by a lactic acid producing bacterial strain for use in the treatment or prevention of child attachment disorder, wherein the lactic acid producing bacterial strain, the conditioned medium and/or the microvesicles is or are capable of inducing release of secretin locally in a gastrointestinal tract of a subject suffering from child attachment disorder or having the risk of suffering from child attachment disorder. In such a case, the locally released secretin is further capable of inducing production and/or release of oxytocin locally in the gastrointestinal tract of the subject.

In particular, women who are taking PPIs or have been taking PPIs, for instance to treat or prevent heartburn during pregnancy, would benefit from the lactic acid producing bacterial strains, the conditioned medium and/or the microvesicles of the invention.

Experimental studies in breast feeding women have shown that acute physical and mental stress can impair the milk ejection reflex by reducing the release of oxytocin during a feed. If this occurs frequently, it can also reduce milk production by preventing full emptying of the breast at each feed. Stress during and after childbirth may, thus, severely impair breast feeding of the newborn child. It is, thus, a further aspect and object of the invention to use a lactic acid producing bacterial strain capable of inducing release of secretin to prevent stress-induced breast-feeding problems and to improve emptying of the breast during feeds, and thereby improving milk production in women during and after childbirth.

Yet another aspect, thus, relates to a probiotic composition comprising a lactic acid producing bacterial strain, a conditioned medium from a lactic acid producing bacterial strain and/or microvesicles produced by a lactic acid producing bacterial strain for use in the treatment or prevention of stress-induced breast-feeding problems, e.g., milk ejection reflex impairment and/or breastfeeding with dysphoric milk ejection reflect (D-MER), wherein the lactic acid producing bacterial strain, the conditioned medium and/or the microvesicles is or are capable of inducing release of secretin locally in a gastrointestinal tract of a subject suffering from stress-induced breast-feeding problems or having the risk of suffering from stress-induced breast-feeding problems. In such a case, the locally released secretin is further capable of inducing production and/or release of oxytocin locally in the gastrointestinal tract of the subject.

The subjects in this embodiment are preferably women who have given birth, and in particular women who have given birth within last 12 months, such as within the last 9 months or the last 6 months. In particular, women who have given birth and are taking PPIs or have been taking PPIs, for instance to treat or prevent heartburn during pregnancy, would benefit from the lactic acid producing bacterial strains of the invention.

Lactic acid producing bacterial strains selected according to the embodiments, and/or conditioned medium and/or microvesicles from such lactic acid producing bacterial strains, can induce the secretion of secretin in the gastrointestinal system of mammals to increase secretin levels above normal, to normalize subnormal secretin levels, or to increase secretin levels to less subnormal levels. In particular, the lactic acid producing bacterial strains selected and used according to the invention, and/or conditioned medium and/or microvesicles from such lactic acid producing bacterial strains, are capable of inducing the release of secretin from S-cells locally in the gastrointestinal tract. The intestinal secretin has an effect locally within the gastrointestinal tract but may also have peripheral effects with regard to the gastrointestinal system. Such peripheral effects can be mediated both through the systemic circulation, or through afferent vagal signaling. Secretin can also cross the blood brain barrier, and secretin, or the downstream events induced by secretin, may reach the central nervous system via the systemic circulation or through the gut-brain axis via the vagus nerve. Accordingly, secretin induced by the lactic acid producing bacterial strains, the conditioned medium and the microvesicles of this invention can be used in the treatment of hyposecretinemia and/or a disease related to secretin deficiency in a subject. Secretin induced by the lactic acid producing bacterial strains, the conditioned medium and/or the microvesicles can also be used to boost secretin levels in a subject where secretin levels are normal, but when it is desired to modulate digestion, to slow down gastric emptying, to increase pH neutralization in the intestine, to increase satiety and/or regulate energy balance and thermogenesis. Secretin induced by the lactic acid producing bacterial strain, the conditioned medium and/or the microvesicles can also be used to increase release of pancreatic enzymes to improve digestion of food in the GI tract.

In addition, increasing levels of secretin locally, even when there is no secretin deficiency, could help reduce symptoms of certain digestive problems, such those associated with excessive (rapid) gastric emptying, increased gastric acid levels, and intestinal inflammation. Increased levels of secretin could also have an effect on satiety and inducing secretin production and release by the administration of a probiotic composition capable of the invention could increase satiety, decrease food intake as well as regulate energy balance and thermogenesis.

Taken together, there is a need for safe, efficient, and uncomplicated treatment that can increase secretin levels in subjects in need thereof, such as subjects who would benefit from an increase in secretin levels locally in the gastrointestinal tract or subjects with an overall secretin deficiency. Especially subjects suffering from achlorhydria or hypochlorhydria would benefit from a local GI increase in secretin induced by a lactic acid producing bacterial strain, since the endogenous signal for secretin is lost or significantly reduced in subjects where hydrochloric acid is absent or reduced. Administration of a secretin-inducing lactic acid producing bacterial strain, or a conditioned medium or microvesicles from such secretin-inducing lactic acid producing bacterial strain, could, thus, be used in treatment of secretin deficiency and diseases, disorders or conditions related to secretin deficiency. Administration of a secretin-inducing lactic acid producing bacterial strain would in particular be beneficial in subjects suffering from achlorhydria-and/or hypochlorhydria-dependent digestive dysfunction. The present invention responds to this need, providing products that can safely and effectively prevent and treat secretin deficiency and diseases associated with this deficiency.

In another embodiment of the current invention, the lactic acid producing bacterial strains, conditioned medium and/or microvesicles can be used to induce the secretion of secretin in the gastrointestinal tract or system of a subject in order to increase satiety. Secretin is a known anorectic peptide and signals peripheral satiety via the vagal afferent or by crossing the blood-brain-barrier. Administration of a secretin-inducing lactic acid producing bacterial strain, conditioned medium and/or microvesicles could, thus, dually increase satiety. Use of a lactic acid producing bacterial strain, conditioned medium and/or microvesicles for local induction of secretin in the gastrointestinal tract, could increase satiety by i) a vagal anorectic signal to the central nervous system, ii) by reduced gastric emptying, and iii) by secretin binding to brown adipose tissue to stimulate thermogenesis. This would be especially important for subjects suffering from hyperphagia to reduce their food intake, but also to prevent or treat obesity if the secretin-inducing bacterial strain, conditioned medium and/or microvesicles is or are administered pre-meal to increase satiety and reduce food-intake.

Accordingly, the invention also relates to a probiotic composition comprising a lactic acid producing bacterial strain, a conditioned medium from a lactic acid producing bacterial strain and/or microvesicles produced by a lactic acid producing bacterial strain for use in treatment or prevention of obesity, wherein the lactic acid producing bacterial strain, the conditioned medium and/or the microvesicles is or are capable of inducing release of secretin locally in a gastrointestinal tract of a subject suffering from obesity or having a risk of suffering from obesity. A related use of the probiotic composition is for use in increasing satiety, in particular in a subject suffering from an eating disorder or hyperphagia.

In an embodiment, the lactic acid producing bacterial strain, conditioned medium and/or microvesicles in the above-mentioned various medical uses is or are not only capable of inducing release of secretin but is capable of inducing production and release of secreting locally in the gastrointestinal tract of a subject. Lactic acid producing bacterial strains, also referred to as lactic acid bacterial strains, are gram-positive, low-GC, acid-tolerant, generally non-sporulating, non-respiring and either rod-shaped or spherical bacteria that are capable of producing lactic acid as the major metabolic end product of carbohydrate fermentation. Lactic acid producing bacteria grow anaerobically, but unlike most anaerobes, they are capable of growing also in the presence of oxygen as aerotolerant, or relative oxygen-tolerant anaerobes. Lactic acid producing bacteria are generally recognized as safe (GRAS) and include genera in the order Lactobacillies, which includes Lactobacillus, Leuconostoc, Pediococcus, Lactococcus and Streptococcus, in addition to Aerococcus, Carnobacterium, Enterococcus, Oenococcus, Tetragenococcus, Vagococcus, and Weissella, and also the genera Bifidobacterium in the order of Bifidobacteriales.

In the last decades new analytical tools have enabled scientists to discover many new bacterial species as well as realizing that the species historically grouped under Lactobacillus were too different from each other. To keep the probiotic groups accurate and organized, the genus Lactobacillus was therefore split into 25 different genera. As a result, many probiotics have recently been given new genus names. Therefore, an alternative genus name for Lactobacillus reuteri is Limosilactobacillus reuteri.

In an embodiment, the lactic acid producing bacterial strain used according to the invention is a Lactobacillus bacterial strain. In a particular embodiment, the Lactobacillus bacterial strain a Lactobacillus reuteri bacterial strain.

In an embodiment, the L. reuteri bacterial strain capable of inducing release of secretin locally in the gastrointestinal tract of a subject is selected from the group consisting of L. reuteri ATCC 6475, L. reuteri ATCC 4659, L. reuteri ATCC 5289, L. reuteri DSM 33632, L. reuteri DSM 33633, L. reuteri DSM 33634and L. reuteri DSM 33635, and any combination thereof.

Lactobacillus reuteri ATCC PTA 6475 was deposited under the Budapest Treaty at the American Type Culture Collection (10801 University Blvd., Manassas, VA 20110-2209, U.S.) on Dec. 21, 2004.

Lactobacillus reuteri ATCC PTA 4659 was deposited under the Budapest Treaty at the American Type Culture Collection (10801 University Blvd., Manassas, VA 20110-2209, U.S.) on Sep. 11, 2002.

Lactobacillus reuteri ATCC PTA 5289 was deposited under the Budapest Treaty at the American Type Culture Collection (10801 University Blvd., Manassas, VA 20110-2209, U.S.) on Jun. 25, 2003.

Lactobacillus reuteri DSM 33632, DSM 33633, DSM 33634, and DSM 33635 were deposited under the Budapest Treaty at the Leibniz Institut DSMZ-Deutsche Sammlung von Mikroorganismen und Zellkulturen GmbH (Inhoffenstr. 7B, D-38124 Braunschweig, Germany) on Sep. 9, 2020.

In a particular embodiment, the L. reuteri bacterial strain capable of inducing release of secretin locally in the gastrointestinal tract of a subject is selected from the group consisting of L. reuteri ATCC PTA 6475, L. reuteri ATCC PTA 4659, L. reuteri ATCC 5289, L. reuteri DSM 33633, L. reuteri DSM 33635, and any combination thereof.

In another particular embodiment, the L. reuteri bacterial strain capable of inducing release of secretin locally in the gastrointestinal tract of a subject is selected from the group consisting of L. reuteri ATCC 6475 and L. reuteri ATCC 4659, and any combination thereof.

In a further particular embodiment, the L. reuteri bacterial strain capable of inducing release of secretin locally in the gastrointestinal tract of a subject is selected from the group consisting of L. reuteri ATCC 6475, L. reuteri ATCC 4659, and L. reuteri ATCC 5289, and any combination thereof.

In yet another particular embodiment, the L. reuteri bacterial strain capable of inducing release of secretin locally in the gastrointestinal tract of a subject is selected from the group consisting of L. reuteri DSM 33632, L. reuteri DSM 33633, L. reuteri DSM 33634 and L. reuteri DSM 33635, and any combination thereof.

In an another particular embodiment, the L. reuteri bacterial strain capable of inducing release of secretin locally in the gastrointestinal tract of a subject is selected from the group consisting of L. reuteri DSM 33632 and L. reuteri DSM 33633, and any combination thereof.

In another particular embodiment, the L. reuteri bacterial strain capable of inducing release of secretin locally in the gastrointestinal tract of a subject is selected from the group consisting of L. reuteri DSM 33634 and L. reuteri DSM 33635, and any combination thereof.

In a further particular embodiment, the L. reuteri bacterial strain capable of inducing release of secretin locally in the gastrointestinal tract of a subject is selected from the group consisting of L. reuteri ATCC 5289, L. reuteri DSM 33633, L. reuteri DSM 33635, and any combination thereof.

In yet another particular embodiment, the L. reuteri bacterial strain capable of inducing release of secretin locally in the gastrointestinal tract of a subject is selected from the group consisting of L. reuteri DSM 33633 and L. reuteri DSM 33635, and any combination thereof.

In an embodiment, the L. reuteri strain is L. reuteri ATCC PTA 6475.

In another embodiment, the L. reuteri strain is L. reuteri ATCC PTA 4659.

In a further embodiment, the L. reuteri strain is L. reuteri ATCC PTA 5289.

In yet another embodiment, the L. reuteri strain is L. reuteri DSM 33632.

In another embodiment, the L. reuteri strain is L. reuteri DSM 33633.

In a further embodiment, the L. reuteri strain is L. reuteri DSM 33634.

In yet another embodiment, the L. reuteri strain is L. reuteri DSM 33635.

In an embodiment, the L. reuteri bacterial strain is other than L. reuteri ATCC 6475. In an embodiment, the L. reuteri bacterial strain is other than L. reuteri ATCC 4659. In an embodiment, the L. reuteri bacterial strain is other than L. reuteri ATCC 6475 and L. reuteri ATCC 4659.

In an embodiment, the probiotic composition comprises the lactic acid producing bacterial strain capable of inducing release of secretin locally in a gastrointestinal tract of a subject.

In another embodiment, the probiotic composition comprises the conditioned medium capable of inducing release of secretin locally in a gastrointestinal tract of a subject. In a particular embodiment, the conditioned medium is from a lactic acid producing bacterial strain capable of inducing release of secretin locally in a gastrointestinal tract of a subject.

In a further embodiment, the probiotic composition comprises the microvesicles capable of inducing release of secretin locally in a gastrointestinal tract of a subject. In a particular embodiment, the microvesicles are produced by a lactic acid producing bacterial strain capable of inducing release of secretin locally in a gastrointestinal tract of a subject.

It is also possible to provide probiotic compositions comprising combinations of the lactic acid producing bacterial strain, the conditioned medium and the microvesicles. Hence, in further embodiments, the probiotic composition comprises a lactic acid producing bacterial strain capable of inducing release of secretin locally in a gastrointestinal tract of a subject and a conditioned medium capable of inducing release of secretin locally in the gastrointestinal tract of the subject; the probiotic composition comprises a lactic acid producing bacterial strain capable of inducing release of secretin locally in a gastrointestinal tract of a subject and microvesicles capable of inducing release of secretin locally in the gastrointestinal tract of the subject; the probiotic composition comprises a conditioned medium capable of inducing release of secretin locally in a gastrointestinal tract of a subject and microvesicles capable of inducing release of secretin locally in the gastrointestinal tract of the subject; or the probiotic composition comprises a lactic acid producing bacterial strain capable of inducing release of secretin locally in a gastrointestinal tract of a subject, a conditioned medium capable of inducing release of secretin locally in the gastrointestinal tract of the subject and microvesicles capable of inducing release of secretin locally in the gastrointestinal tract of the subject.

In these embodiments, the conditioned medium and/or the microvesicles are from the same lactic acid producing bacterial strain included in the probiotic composition or is or are from another lactic acid producing bacterial strain capable of inducing release of secretin locally in a gastrointestinal tract of a subject.

A probiotic composition comprising the conditioned medium and/or the microvesicles but not necessarily the lactic acid producing bacterial strain may be regarded as a postbiotic product. Such a postbiotic product is defined as a “preparation of inanimate microorganisms and/or their components that confers a health benefit on the host”. Examples of a postbiotic product according to the invention is, for example, (but not limited to) supernatant (conditioned medium) obtained from cultured lactic acid producing bacteria and comprising metabolites, such as proteins or peptides, or other structures released from the lactic acid producing bacteria during culture, and microvesicles produced by the lactic acid producing bacteria.

A preferred mode of administration of the probiotic composition is oral. Other modes of administration include nasal, intraocular, topical or some other form of local administration to the skin, rectum, nose, eyes, vagina, or gums.

Appropriate doses of the probiotic composition as defined herein can readily be chosen depending on the disease or condition related to hyposecretinemia to be treated, the mode of administration, the subject and the formulation concerned. For example, a dosage and administration regime are chosen such that the probiotic composition administered to the subject in accordance with the present invention can result in desired therapeutic effects, prophylactic effects, or health benefits. Thus, preferably the dosage is a therapeutically or prophylactically effective dosage, which is appropriate for the type of subject and hyposecretinemia being treated. For example, daily doses of 10⁴ to 10¹¹, for example 10^{5 to} 10⁹, or 10⁶ to 10⁸, or 10⁸ to 10¹⁰ total colony forming units (CFUs) of lactic acid producing bacteria may be used. A preferred daily dose is around 10⁸ total CFUs, e.g., 10⁷ to 10⁹ or 10⁸ to 10⁹ CFUs. The probiotic composition can be administered together with food or before or after food intake. In one embodiment the probiotic composition are administered before food intake, for example at least one hour, at least 30 minutes, or at least 15 minutes before food intake.

In one embodiment the daily dose (e.g., 10⁵ to 10⁹ or 10⁶ to 10⁸ or 10⁸ to 10¹⁰ or 10¹⁰ to 10¹² of CFUs) is divided into several administrations e.g., 2-8 administrations (such as 3-5 administrations) or in association with every food intake of the subject. In one embodiment, a higher dose, such as 2 to 10 or 10 to 100 times the dose after the starting period, and/or more frequently administered dose is administered as a starting dose for 3-14 days (e.g., 5-10 days, such as 7 days) to build up the hormone level in a subject.

The lactic acid producing bacterial strain is preferably administered in a pure, isolated, dried, lyophilized or freeze-dried form. The lactic acid producing bacteria can also be administered in a frozen or active formulation (e.g., in a fermented food product). Hence, the lactic acid producing bacterial strain is preferably produced or prepared in a lyophilized or freeze-dried form.

The lactic acid producing bacteria comprised in the probiotic composition are preferably comprised in the probiotic composition as dried, such as lyophilized or freeze-dried, spray-dried or spray-freeze dried or vacuum-dried bacteria.

The probiotic composition may comprise, in addition to the lactic acid producing bacterial strain, the conditioned medium and/or the microvesicles, at least one excipient and/or another active agent. Non-limiting, but illustrative, examples of such excipients include fillers, anti-adherents, binders, coatings, colors, disintegrants, flavors, glidants, lubricants, preservatives, sorbents, sweeteners, and vehicles.

Other active agents that could be comprised in the probiotic composition include such active agents that are used for treatment of any of the above-mentioned examples of secretin deficiency.

The probiotic composition is advantageously administered as a food supplement, formulated as a tablet, capsule, liquid-based formulation such as oil drops, or as a powder.

The subject to be treated with the probiotic composition is a mammal or bird, and preferably a human. The lactic acid producing bacterial strain or composition could, however, also or alternatively be used for veterinary purposes. In such a case, the subject could, for instance, be selected among cats, dogs, sheep, goats, cows, horses, pigs, birds as illustrative, but non-limiting, examples.

The invention also relates to a method for treating or preventing hyposecretinemia. The method comprises administering, to a subject suffering from hyposecretinemia or having a risk of suffering from hyposecretinemia, a probiotic composition comprising lactic acid producing bacterial strain, a conditioned medium from a lactic acid bacterial strain and/or microvesicles produced from a lactic acid bacterial strain, wherein the lactic acid producing bacterial strain, the conditioned medium and/or the microvesicles is or are capable of inducing release of secretin locally in a gastrointestinal tract of the subject.

The invention further defines a method for treating or preventing child attachment disorder. The method comprises administering, to a subject, preferably a female subject, suffering from child attachment disorder, a probiotic composition comprising lactic acid producing bacterial strain, a conditioned medium from a lactic acid bacterial strain and/or microvesicles produced from a lactic acid bacterial strain, wherein the lactic acid producing bacterial strain, the conditioned medium and/or the microvesicles is or are capable of inducing release of secretin locally in a gastrointestinal tract of the subject.

The invention also defines a method for treating or preventing stress-induced breast-feeding problems, e.g., milk ejection reflex impairment and/or breastfeeding with dysphoric milk ejection reflect (D-MER). The method comprises administering, to a subject, preferably a female subject, suffering from stress-induced breast-feeding problems, a probiotic composition comprising lactic acid producing bacterial strain, a conditioned medium from a lactic acid producing bacterial strain and/or microvesicles produced from a lactic acid producing bacterial strain, wherein the lactic acid producing bacterial strain, the conditioned medium and/or the microvesicles is or are capable of inducing release of secretin locally in a gastrointestinal tract of the subject.

The invention additionally defines a method for treating or preventing obesity. The method comprises administering, to a subject suffering from obesity or having a risk of suffering from obesity, probiotic composition comprising lactic acid producing bacterial strain, a conditioned medium from a lactic acid producing bacterial strain and/or microvesicles produced from a lactic acid producing bacterial strain, wherein the lactic acid producing bacterial strain, the conditioned medium and/or the microvesicles is or are capable of inducing release of secretin locally in a gastrointestinal tract of the subject.

The invention also defines a method for increasing satiety. The method comprises administering, to a subject suffering from an eating disorder or hyperphagia, a probiotic composition comprising lactic acid producing bacterial strain, a conditioned medium from a lactic acid producing bacterial strain and/or microvesicles produced from a lactic acid producing bacterial strain, wherein the lactic acid producing bacterial strain, the conditioned medium and/or the microvesicles is or are capable of inducing release of secretin locally in a gastrointestinal tract of the subject. “Treating” or “treatment” as used herein and is well understood in the art, means an approach for obtaining beneficial or desired results, including clinical results. Beneficial or desired clinical results could include, for instance, alleviation or amelioration of one or more symptoms or conditions, diminishment of extent of hyposecretinemia, stabilized state of secretin deficiency, i.e., prevent worsening, delay or slowing of disease progression, amelioration or palliation of the hyposecretinemia state, diminishment of the reoccurrence of hyposecretinemia, and remission. “Treating” or “treatment” may also prolong survival as compared to expected survival if not receiving any treatment. Treatment of hyposecretinemia as used herein also encompasses inhibiting hyposecretinemia in a subject and prophylactic treatment. “Preventing” or “prophylaxis” or “reducing the risk” as used herein and is well understood in the art, means an approach in which a risk of developing hyposecretinemia is reduced or prevented, including prolonging or delaying hyposecretinemia development. For instance, a patient predisposed to develop hyposecretinemia, such as due to genetic or hereditary predisposition, could benefit for administration of the lactic acid producing bacterial strains of the embodiments to prevent, reduce the risk of, delaying and/or slowing development of the hyposecretinemia.

### EXAMPLES

### Example 1

This example investigated the capability of various lactic acid producing bacterial strains in inducing secretin release from enteroid cells.

### Materials and Methods

### Enteroid Cell Line

A cell line genetically engineered to increase the number of enteroendocrine cells present in the culture from ≤1% to ˜40% was used. This enteroid cell line is obtained from human jejunum (“J2-tetNGN3”) and consists of intestinal enteroendocrine cells. Information of the enteroid cell line, including culturing conditions and general assay methodology is presented in Cellular and Molecular Gastroenterology and Hepatology (2019) 8(2): P209-229.

### Media

Lactobacilli MRS broth (Difco), sterilized by autoclaving for 15 minutes at 121° C., was used for overnight culturing.

LDM4—minimal media produced as previously described (Frontiers in Microbiology (2019) 10: Article 2305), filter sterilized, was used for sub-culturing to prepare supernatants to use on the enteroid cell lines. Krebs-Ringer Bicarbonate Buffer (Sigma Aldrich).

### Filters

Large Volume: Thermo Scientific Nalgene Rapid Flow 0.22 μM filters.

Small volume: Miliplex Millex GV Low protein binding (PVDF) Syringe filters.

### Culture Plates

Sterile Multi-well 6 well polystyrene flat bottom, non-tissue culture treated plates (Falcon) were used for tissue assays.

### Reagents

Secretin ELISA Kit (Biomatik) (multiple lots used) was used according to manufacturer's instructions.

Oxytocin ELISA Kit (Enzo Life Sciences) (multiple lots used) was used according to manufacturer's instructions.

### Bacterial Culture

In this Example, L. reuteri ATCC PTA 6475 was tested.

Living L. reuteri bacteria may, if added directly to the culture, cause a rapid acidification in the glucose-rich culture environment. Such an acidic environment may be deleterious to the enteroid cell line causing stress or may even be lethal. Accordingly conditioned LDM4 medium was produced and added to the enteroid cell line.

In more detail, L. reuteri cells were cultured in MRS for approximately 16 hours in a 37° C. incubator. The culture was mixed by inversion until the cell pellet was homogenously resuspended. The culture was diluted 1:10 and the optical density (OD) at 600 nm was read by spectrophotometer. Then, a 40 ml of pre-warmed LDM 4 medium was inoculated at a starting OD of 0.1 and was placed into a 37° C. water bath to incubate for ˜5 hours, until the OD reached mid exponential phase (0.5) The L. reuteri cells were pelleted by centrifugation at 4700 rpm and the supernatant was transferred to a new 50 ml conical tube. The supernatant was pH neutralized (pH 7.0) using 10-30 μl of sodium hydroxide solution and was sterilized by 0.22 μm filter sterilization.

A total volume of 100 μL conditioned medium (sterilized supernatant) was added to the enteroid cells and incubated for 3 hours at 37° C., 5% CO₂ in a tissue culture incubator. After the incubation, the supernatant was removed and stored at −20° C. until analysis.

### Analysis

Secretin and oxytocin in the supernatant thawed and tempered to room temperature (20-21° C.) were detected by ELISA according to the manufacturers'protocol.

### Results

As shown in FIG. 1, conditioned medium from L. reuteri ATCC PTA 6475 increased the secretin levels from the enteroid cells compared to the control medium (LDM4), in which no secretin was detected.

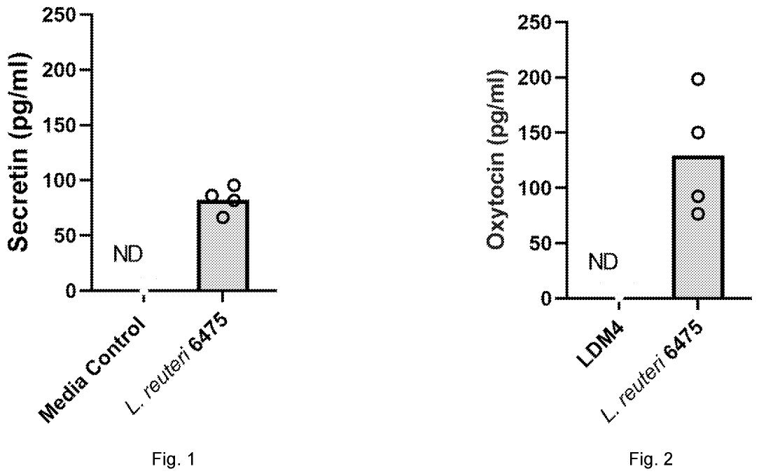

Also, conditioned medium from L. reuteri ATCC PTA 6475 increased the oxytocin levels from the enteroid cells compared to the control medium (LDM4) where no oxytocin was detected (FIG. 2).

L. reuteri ATCC PTA 6475 was, thus, selected as a lactic acid producing bacterial strain capable of inducing release of secretin locally in the gastrointestinal tract and is therefore useful in the treatment of hyposecretinemia. In the same way, lactic acid producing bacterial strains found to be capable of inducing oxytocin would be selected for treatment purposes related to reduced or subnormal levels of oxytocin.

### Example 2

This example investigated the capability of various lactic acid producing bacterial strains in inducing secretin release from ex vivo intestine tissue.

### Materials and Methods

### Bacterial Culture

In this experiment two different strains of Lactobacillus reuteri were tested: L. reuteri ATCC PTA 6475 and 4659

Human intestines were acquired through the organ donation group LifeGift (Houston, TX, USA). Whole intestines were delivered on ice within 1 hour of removal. Intestinal regions were excised (sizes between 1.4 to 8cm² ), washed three times in cold sterile phosphate-buffered saline (PBS), and used for secretion assays.

5 ml of pH neutralized filter sterilized L. reuteri conditioned medium prepared as in Example 1 was placed in a 6 well plate. Plates containing medium were prewarmed in a 37° C. 5% CO₂ tissue culture incubator while intestinal tissue was dissected and washed. The tissue sections were submerged in the conditioned medium and incubated for 3 hours in 5% CO₂. The tissue sections were removed and the supernatant was transferred to a 15 ml conical tube with a single use 5 ml serological pipette (Greiner Bio-one).

Debris was collected by centrifugation at 4700 rpm and the supernatant was aliquoted for storage at −20° C. for downstream analysis.

### Analysis

Secretin in the supernatant thawed and tempered to room temperature (20-21° C.) were detected by ELISA according to the manufacturer's protocol. The concentration of secretin was normalized to the area of the tissue section.

### Results

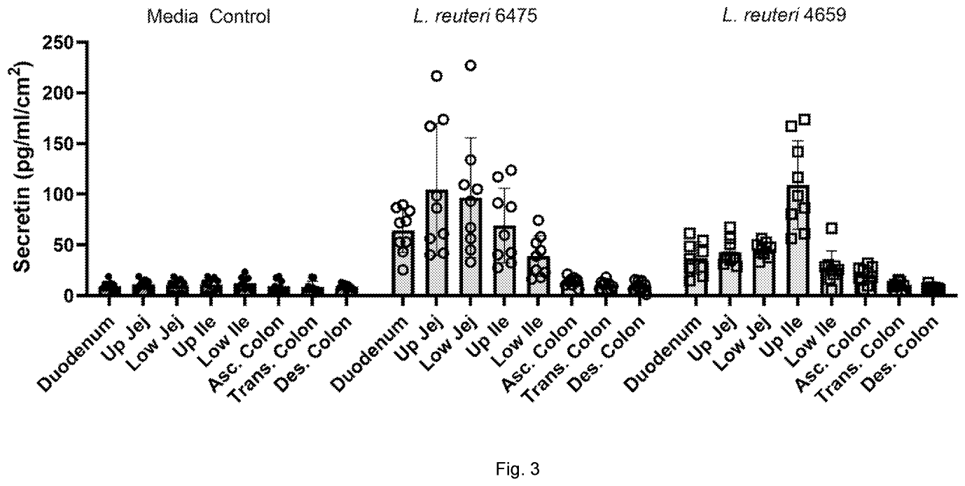

As shown in FIG. 3, conditioned medium from L. reuteri ATCC PTA 6475 and 4659 increased the secretin levels from the intestinal tissue compared to the control medium (LDM4). Both L. reuteri ATCC PTA 6475 and 4659 were thus, selected as a lactic acid producing bacterial strains capable of inducing release of secretin locally in the gastrointestinal tract and they are therefore useful in the treatment of hyposecretinemia.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1. A method of treating or preventing hyposecretinemia in a subject, comprising administering to the subject a probiotic composition comprising a lactic acid producing bacterial strain, a conditioned medium from a lactic acid producing bacterial strain and/or microvesicles produced by a lactic acid producing bacterial strain wherein the lactic acid producing bacterial strain, the conditioned medium and/or the microvesicles induce release of secretin locally in a gastrointestinal tract of a subject suffering from hyposecretinemia or having a risk of suffering from hyposecretinemia.

2. The method according to claim 1, wherein
the subject suffers from hyposecretinemia caused by achlorhydria and/or hypochlorhydria or has a risk of suffering from hyposecretinemia caused by achlorhydria or hypochlorhydria; and
the lactic acid producing bacterial strain, the conditioned medium and/or the microvesicles induce release of secretin locally in the gastrointestinal tract of the subject suffering from hyposecretinemia caused by achlorhydria or hypochlorhydria or having a risk of suffering from hyposecretinemia caused by achlorhydria or hypochlorhydria.

3. The method according to claim 2, wherein the subject is an elderly subject suffering from achlorhydria or hypochlorhydria.

4. The method according to claim 2, wherein the method treats or prevents achlorhydria-dependent or hypochlorhydria-dependent dysfunctional digestion in a subject suffering from achlorhydria-dependent or hypochlorhydria-dependent dysfunctional digestion or having a risk of suffering from achlorhydria-dependent or hypochlorhydria-dependent dysfunctional digestion.

5. The method according to claim 2, wherein the subject is taking a medication for lowering gastric acid production and/or release, preferably a proton-pump inhibitor (PPI) and/or a histamine receptor antagonist (H2 blocker).

6. The method according to claim 5, wherein the subject is a pregnant woman taking a PPI and/or a H2 blocker.

7. The method according to claim 5, wherein the method treats or prevents adverse effects associated with taking a PPI and/or a H2 blocker.

8. The method according to claim 1, wherein the subject is suffering from a condition causing achlorhydria or hypochlorhydria, wherein the condition is selected from the group consisting of chronic gastritis, atrophic gastritis, chronic stress, bacterial infection, alcohol overconsumption, or vitamin deficiency, or wherein the subject has undergone a gastric surgery and/or a vagotomy.

9. The method according to claim 1, wherein the subject is suffering from malnutrition.

10. The method according to claim 1, wherein the lactic acid producing bacterial strain is a Lactobacillus reuteri bacterial strain that induces release of secretin locally in the gastrointestinal tract of the subject.

11. The method according to claim 10, wherein the L. reuteri bacterial strain is selected from the group consisting of L. reuteri ATCC PTA 6475, L. reuteri ATCC PTA 4659, L. reuteri ATCC 5289, L. reuteri DSM 33632, L. reuteri DSM 33633, L. reuteri DSM 33634, L. reuteri DSM 33635, and any combination thereof.

12. The method according to claim 11, wherein the L. reuteri bacterial strain is selected from the group consisting of L. reuteri ATCC PTA 6475, L. reuteri ATCC PTA 4659, L. reuteri ATCC 5289, L. reuteri DSM 33633, L. reuteri DSM 33635, and any combination thereof, or
selected from the group consisting of L. reuteri ATCC PTA 6475, L. reuteri ATCC PTA 4659, and any combination thereof, or
selected from the group consisting of L. reuteri ATCC 5289, L. reuteri DSM 33633, L. reuteri DSM 33635, and any combination thereof.

13-14. (canceled)

15. The method according to claim 12, wherein the L. reuteri bacterial strain is L. reuteri L. reuteri DSM 33635.

16. A method for selecting a lactic acid producing bacterial strain, the method comprising:
contacting ex vivo intestinal enteroid cells, enteroendocrine S cells or gastrointestinal tissue with a lactic acid producing bacterial strain and/or a conditioned medium from the lactic acid producing bacterial strain;
detecting any secretin released from the intestinal enteroid cells, enteroendocrine S cells or gastrointestinal tissue in response to contact with the lactic acid producing bacterial strain and/or the conditioned medium; and
selecting the lactic acid producing bacterial strain if the lactic acid producing bacterial strain and/or the conditioned medium is determined to be capable of inducing secretin release from the intestinal enteroid cells, enteroendocrine S cells or gastrointestinal tissue.

17. The method according to claim 16, further comprising:
detecting any secretin released from the intestinal enteroid cells, enteroendocrine S cells or gastrointestinal tissue in response to contact with a reference medium lacking the lactic acid producing bacterial strain and the conditioned medium; and
comparing an amount of secretin released from the intestinal enteroid cells, enteroendocrine S cells or gastrointestinal tissue in response to contact with the lactic acid producing bacterial strain and/or the conditioned medium with an amount of secretin released from the intestinal enteroid cells, enteroendocrine S cells or gastrointestinal tissue in response to contact with the reference medium, wherein
selecting the lactic acid producing bacterial strain comprises selecting the lactic acid producing bacterial strain if the amount of secretin released from the intestinal enteroid cells, enteroendocrine S cells or gastrointestinal tissue in response to contact with the lactic acid producing bacterial strain and/or the conditioned medium is higher than the amount of secretin released from the intestinal enteroid cells, enteroendocrine S cells or gastrointestinal tissue in response to contact with the reference medium.

18. The method according to claim 16, wherein selecting the lactic acid producing bacterial strain comprises selecting the lactic acid producing bacterial strain as suitable for use in treatment or prevention of hyposecretinemia if the lactic acid producing bacterial strain and/or the conditioned medium is determined to be capable of inducing secretin release from the intestinal enteroid, enteroendocrine S cells or gastrointestinal tissue.

19. A method for selecting a lactic acid producing bacterial strain, the method comprising:
feeding a germ-free subject with a lactic acid producing bacterial strain and/or a conditioned medium from the lactic acid producing bacterial strain;
detecting, in a body sample taken from the germ-free subject, any secretin released from gastrointestinal tissue of the subject induced by the lactic acid producing bacterial strain and/or the conditioned medium; and
selecting the lactic acid producing bacterial strain if secretin released from the gastrointestinal tissue of the germ-free subject induced by the lactic acid producing bacterial strain and/or the conditioned medium is detected.

20. The method according to claim 19, further comprising:
feeding a germ-free reference subject with a reference medium lacking the lactic acid producing bacterial strain and the conditioned medium from the lactic acid producing bacterial strain;
detecting, in a body sample taken from the germ-free reference subject, any secretin released from the gastrointestinal tissue of the germ-free reference subject induced by the reference medium; and
comparing an amount of secretin released from the gastrointestinal tissue of the germ-free subject induced by the lactic acid producing bacterial strain and/or the conditioned medium with an amount of secretin released from the gastrointestinal tissue of the germ-free reference subject induced by the reference medium, wherein
selecting the lactic acid producing bacterial strain comprises selecting the lactic acid producing bacterial strain if the amount of secretin released from the gastrointestinal tissue of the germ-free subject induced by the lactic acid producing bacterial strain and/or the conditioned medium is higher than the amount of secretin released from the gastrointestinal tissue of the germ-free reference subject induced by the reference medium.

21. The method according to claim 19, wherein selecting the lactic acid producing bacterial strain comprises selecting the lactic acid producing bacterial strain as suitable for use in treatment or prevention of hyposecretinemia if secretin released from the gastrointestinal tissue of the germ-free subject induced by the lactic acid producing bacterial strain and/or the conditioned medium is detected.

22. The method according to claim 16, wherein the lactic acid producing bacterial strain is a Lactobacillus reuteri bacterial strain.
